(19) United States
(12) Patent Application Publication
(10) Pub. No.: US 2004/0092837 A1
(43) Pub. Date: May 13, 2004

(54) Dual frequency transmitter

(75) Inventor: Callum MacGregor, Chichester (GB)

Correspondence Address: Jefferson Perkins, PIPER RUDNICK, P.O. Box 64807, Chicago, IL, 60664-0807, US

(73) Assignee: PULSE TIME PRODUCTS LIMITED

(21) Appl. No.: 10/376,755

(22) Filed: Feb. 28, 2003

(30) Foreign Application Priority Data
Nov. 9, 2002 (GB) 0226208.7

(51) Int. Cl. A61B 5/0402

(52) U.S. Cl. 600/519; 600/520

(57) ABSTRACT

A dual frequency transmitter, for use in remote monitoring of the heart rate of a living animal or human body, emits a first radio signal having a frequency of about 5 KHz and a second radio signal having a frequency substantially greater than 5 KHz. The dual frequency transmitter is preferably incorporated into personal property of the user, such as a wristwatch.

The present invention relates to dual frequency transmitting equipment used in the remote monitoring of the heart rate of a living animal or human body, both in an environment where there are strong electrical disturbances or several heart rate monitoring systems operating in close proximity, and also in a disturbance-free environment.

It is common practice when remotely monitoring the heart rate of animals and humans to place a transmitter on the chest of the subject, and to have a receiver/monitor disposed within one metre or so of the transmitter, for example on the wrist of a human user or on the handlebars of a piece of exercise equipment that the subject is using.

The transmitter, which is commonly mounted on a chestbelt, picks up the electrical activity of the subject's heart by means of conducting contacts under the chestbelt. These signals are amplified and each heartbeat detected by the arrival of each so-called R-wave from the heart. For each detected R-wave the transmitter conventionally emits a simple short burst of radio waves.

The receiver/monitor, if it is sufficiently close to the transmitter, detects each burst of radio waves, times the intervals between bursts and converts these times to a heart rate, suitably averaged. The heart rate is then displayed, and, in more complex systems, may be recorded periodically.

The most commonly used radio frequency in heart rate monitors is 5 kHz (corresponding to a huge wavelength of 60 km), which, for radio, is a very-low frequency indeed; so low that the coupling between transmitter and receiver is better thought of as by very loose magnetic coupling” rather than by propagating radio waves. However, in the radio spectrum in the region of 5 kHz there is often a great deal of electrical disturbance—especially from electromechanical equipment and computer monitors. Consequently, there is very often a problem with electrical interference when heart rate monitors are used in conjunction with exercise equipment that incorporates electric motors or extensive electronic circuitry or refreshed display means. The unfortunate result is that the signal picked up by the receiver/monitor from the chest-worn transmitter becomes swamped by equipment-generated noise, and heart rate monitoring becomes very unreliable.

There is therefore a need for a remote monitoring system which can operate reliably in an environment in which there is a significant level of electrical disturbance.

Furthermore, for any radio signal to be detected reliably, the signal must contain at least several cycles of the radio wave—typically more than ten. At 5 kHz, therefore, the minimum burst duration is a few milliseconds. Systems currently in use generally operate with burst lengths of 3 to 15 ms. The output stage of the transmitter is therefore switched on and consumes considerable current for this period at every heartbeat.

Another disadvantage of known heart rate monitors arises in situations where there is more than one monitoring system operating in close proximity. In such situations, a receiver may receive the signals from more than one transmitter. This is another common serious form of interference which can render monitoring ineffective.

One solution to this problem would appear to be to use an HF or UHF transmitter which would give rise to shorter transmission times due to the higher frequency, and which could more readily be employed to transmit coded signals such as those taught in GB2334650. However, the corresponding HF or UHF receivers would draw rather a high current and would be relatively costly and, although providing satisfactory performance in the electrically noisy environments of a gymnasium, have not been practical for use with consumer heart rate monitors which are more commonly used outdoors or at home, where the likelihood of interference is low.

It is an object of the present invention to obviate or mitigate at least one disadvantage of the prior art.

It is also desirable to provide a low-cost means of personal heart rate monitoring for a user when he/she exercises on electrically noisy equipment, perhaps within a group of similar users, and also for the user when he/she exercises remote from sources of interference using a heart rate monitoring watch or portable module.

This is achieved by providing a dual frequency transmitter for heart rate monitoring which emits a first radio signal having a frequency of about 5 kHz and a second radio signal having a frequency substantially greater than 5 kHz.

Such a dual frequency transmitter would typically be the personal property of a user, who would wear it while exercising in a gymnasium alongside several similar users, and also typically while jogging alone.

The frequency of the second radio signal may be in the range 100 kHz to 3 GHz, for example 122 kHz. The second radio signal may have a frequency in the HF or UHF band. Any suitable HF or UHF frequency can be employed provided the frequency lies within an approved radio band in the country of use. However the geometrical size of the components of personal heart rate monitors is small, restricting the length of radio aerials. Therefore frequencies in the UHF band are preferred, because the length of an effective aerial can be as short as a few centimetres, which can be accommodated within the transmitters.

An advantage of using an HF or UHF frequency is that the operating range of the high frequency system described here can be extended well beyond the range achievable by the 5 kHz system. This can be useful on long bed treadmills, for example.

According to a first aspect of the present invention, there is provided a transmitter for heart rate monitoring, said transmitter having detection means for detecting a heart rate signal, and heart rate signal processing means for processing the detected heart rate signal into a processed signal, and transmitting means for transmitting a first radio signal having a frequency about 5 kHz and for transmitting a second radio signal at a frequency substantially greater than 5 kHz, said first and said second radio signals corresponding to said processed signal.

Preferably, the frequency of the second radio signal is at least 100 kHz. Conveniently, the frequency of the second radio signal is 122 kHz.

Preferably also, the first and second radio signals are transmitted from respective transmitter output stages simultaneously. Alternatively, the first and second radio signals are successively transmitted from their respective output stages.

Preferably, the heart rate signal processing means includes a threshold detection circuit coupled to a pulse generator current for generating an output pulse when the processed signal exceeds a preset amplitude, said output pulse fed to said 5 kHz transmitter output stage and to said high frequency transmitter output stage for emitting said first and said second radio frequency signals simultaneously.

Alternatively, the heart rate signal processing means includes a threshold detection circuit coupled to a microcontroller for generating an output pulse when the processed signal exceeds a preset amplitude, said output pulse being passed to said 5 kHz transmitter output stage, and to said high frequency output stage for emitting said first and said second signals simultaneously.

Preferably, said transmitter is mounted on a chestbelt. Alternatively, said transmitter may be mounted within a headband or wristwatch.

Preferably also, the transmitter includes switch means for allowing a user to select transmission of the first or the second radio signals or both.

According to another aspect of the present invention, there is provided a receiver for receiving a first and a second radio signal from a transmitter for heart rate monitoring, said first, radio signal being about 5 kHz and said second radio signal being substantially greater than 5 kHz, said receiver having radio receiver means for receiving first and second radio signals, and decoding means coupled to said receiving means for providing at least an output pulse for each correctly received signal.

Preferably, the decoding means is a microcontroller for decoding a coded message and for providing a decoded output signal, said decoded output signal being a single electrical pulse or a message containing the identity of the transmitter and current heart rate.

Alternatively, the decoding means is a decoding circuit for generating a single output pulse of a fixed duration for every correctly received signal.

Conveniently, the receiver output is coupled to a microprocessor disposed in a console.

Preferably also, the receiver is combined with the console.

The first and second signals may be emitted simultaneously or successively.

The first signal in the HF or UHF range allows complex messages, perhaps containing calculated heart rate, unit address and check data, to be transmitted within a short duration. This brief output current consumption helps to conserve battery life. Short messages have a lower probability of clashing with messages from other neighbouring users, therefore the incidence of corrupted messages is reduced. In practice, the signals are coded with a unit “address” in a simple manner such that neighbouring systems can operate simultaneously. There are many methods, well known to these appropriately skilled, of sending digital information by radio—for example as described in GB2334650.

A calculated heart rate may be transmitted as a single byte of binary data.

On the other hand, the 5 kHz emission is relatively simple, consisting of a single short burst (duration a few milliseconds) of 5 kHz at every detected heartbeat. This type of signal is used by virtually all the heart rate monitoring watches currently produced, and makes available a wide is selection of monitors to the user.

Thus in a gymnasium the HF or UHF second signals are be received by a compatible receiver incorporated into the console of each piece of exercise equipment and the user's heart rate is displayed on the console display. Where coded signals are employed, input means are provided to enter into the console a code, such as a unit address, associated with the transmitter worn by the user.

However, when exercising alone, the user may select only the first transmitted signal at substantially 5 kHz to drive a simple heart rate monitoring watch or other heart rate monitoring module. This selection would disable transmission of the high frequency signal saving power and prolonging battery life.

Such a dual frequency transmitter permits effective heart rate monitoring in any environment. The second signal provides effective monitoring in electrically noisy environments where users are in close proximity to the complex electronics incorporated into an equipment console, while the first signal provides effective heart rate monitoring in environments where interference is unlikely to arise.

For a better understanding of the present invention and to show more clearly how it may be carried into effect, reference will now be made, by way of example, to the accompanying drawings, in which:

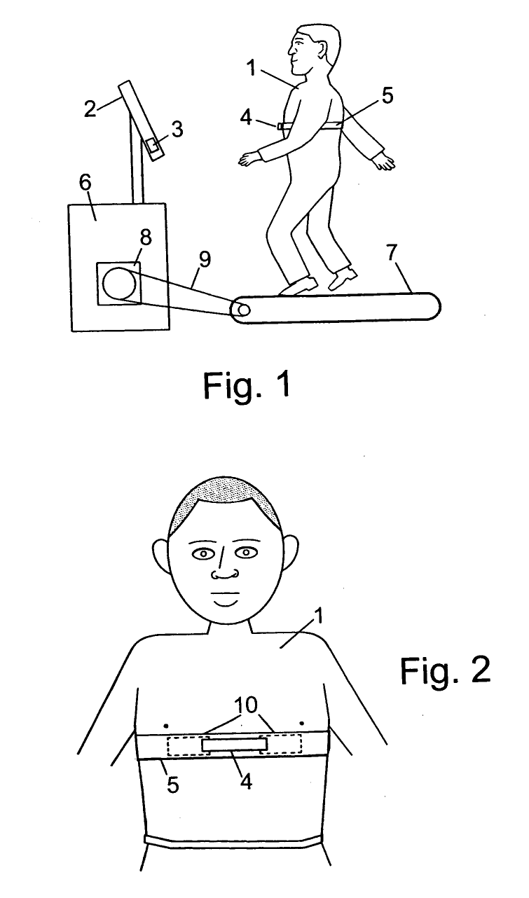

FIG. 1 is a diagrammatic side view of a user with heart rate monitor exercising on a treadmill;

FIG. 2 is a diagrammatic front view of a user wearing the transmitter mounted on a chestbelt;

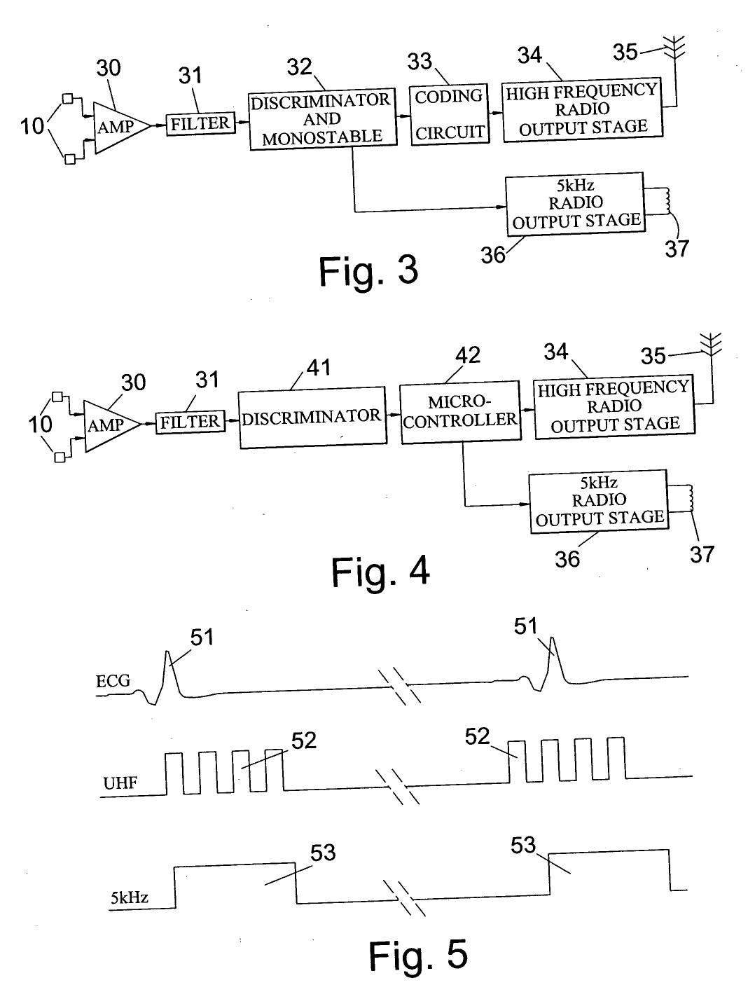

FIG. 3 is a block diagram of the electronic circuitry of a dual frequency transmitter which emits a 5 kHz burst and also a coded UHF signal;

FIG. 4 is a block diagram of the electronic circuitry of a dual frequency transmitter in accordance with a preferred embodiment of the invention which emits a 5 kHz burst, and which also incorporates a microcontroller to generate a more complex coded UHF signal;

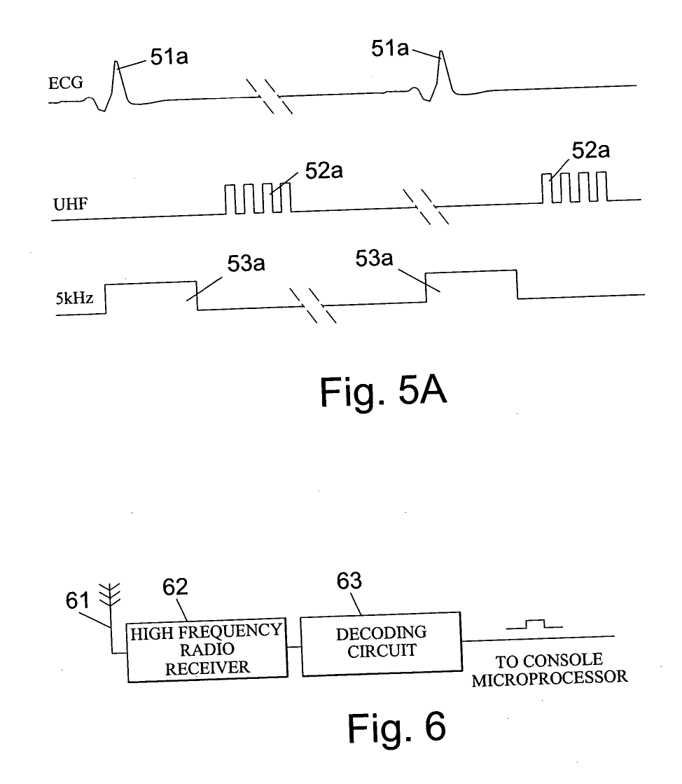

FIG. 5 is a representation of the signals involved in a preferred embodiment of the present invention where the first and second signals are transmitted simultaneously;

FIG. 5A is a representation of the signals involved in a second embodiment of the present invention, where the first and second signals are transmitted sequentially;

FIG. 6 is a block diagram of the electronic circuitry of a UHF receiver which can decode simple codes, that is for use with the transmitter of FIG. 3;

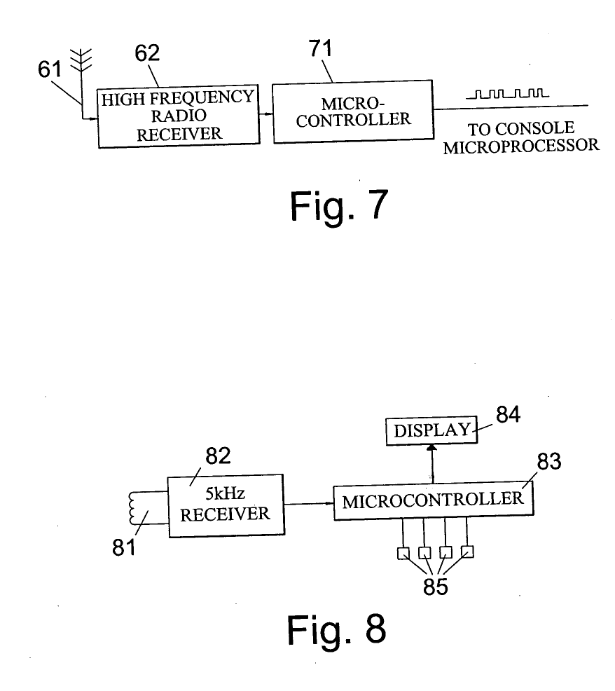

FIG. 7 is a block diagram of the electronic circuitry of a UHF receiver in accordance with a preferred embodiment of the present invention which can decode complex codes, that is for use with the transmitter of FIG. 4; and

FIG. 8 is a block diagram of the electronic circuitry contained within a self-contained monitoring receiver in accordance with a preferred embodiment of the invention such as a watch.

Referring to FIGS. 1 and 2, a human user 1 is exercising on a treadmill 6 by walking on a moving flexible continuous loop mat 7 driven by a motor 8 via a drive belt 9. The user wears a chestbelt 5 on which is mounted a transmitter 4 which picks up the electrical activity of the user's heart by means of conducting contacts 10 which are pressed against the chest of the user. The electronic console 2 of the treadmill contains a HF or UHF radio receiver 3.

FIG. 3 illustrates a first embodiment of a dual transmitter which emits a first radio signal provided by a burst of 5 kHz and an HF or UHF second radio signal at 122 kHz coded with a simple unit address. Its circuitry contains an amplifier 30 to amplify the electrical signals from the heart picked up by contacts 10. These amplified signals are electronically filtered by circuit block 31 to remove extraneous signal components of too high and too low a frequency, and to pass the so-called R-wave of the user's electrocardiogram. Circuit block 32 is in the form of a discriminator and monostable which delivers a short enabling pulse to the 5 kHz radio output stage 36 whenever the filtered signal exceeds a preset threshold amplitude. The 5 kHz signal is transmitted from aerial coil 37.

When triggered by the discriminator and monostable circuit block 32 coding circuitry 33 is also enabled which energizes the high frequency output stage 34 to emit a simple coded pattern of radio waves from aerial 35. typically the coding might simply be a number, corresponding to the code, of closely spaced bursts of HF or UHF radio waves.

FIG. 4 illustrates a second and preferred embodiment of a dual frequency transmitter which emits a burst of 5 kHz and which has the capability of coding an HF or UHF 122 kHz radio signal with a complex message including an address code, optionally with a calculated heart rate and optionally with some check bits. The circuitry of the transmitter includes an amplifier 30 as in the embodiment of FIG. 3 to amplify the electrical signals from the heart picked up by the contacts 10 and filters 31 to remove extraneous signal components of too high or too low a frequency and to pass the R-wave of the user's electrocardiogram.

A discriminator 41 triggers a microcontroller (in a preferred embodiment, a Microchip PIC12C508) 42 whenever the filtered signal exceeds a predetermined threshold amplitude. When triggered, microcontroller 42 delivers a short enabling pulse to the 5 kHz radio output stage 36, the 5 kHZ signal being transmitted from aerial coil 37. Additionally, when triggered, microcontroller 42 formulates a message for transmission by the high frequency output stage 34 by way of the aerial 35, the microcontroller optionally calculating heart rate from timed beat-to-beat intervals.

FIG. 5 illustrates the preferred signals involved within a dual frequency transmitter. The heart's electrical activity in the form of the so-called ECG signal is shown, and in particular the R-wave 51. Detection of each R-wave triggers simultaneously the UHF output signal 52 and the 5 kHz signal 53. The UHF 122 kHz signal depicted is coded with an address and/or data. The 5 kHz signal is a single burst with a duration of a few milliseconds.

FIG. 5A depicts the relative timing of the first and second signals when these are transmitted sequentially. Each signal 52a and 53a is similar to a respective signal shown in FIG. 5. The advantage of sending the signals non-overlapping in time is that the peak current drain from the transmitter's battery is reduced, thereby allowing a wider selection of battery types.

FIG. 6 depicts an HF or UHF receiving module for use in conjunction with the simple transmitter of FIG. 3. Signals captured by aerial 61 are tuned and amplified by radio receiver 62 which feeds its output to a hardware decoding circuit 63 which generates a single electrical pulse of fixed duration for every correctly received signal. In practice the output from this module drives an input of the exercise equipment console's microprocessor.

FIG. 7 is a block diagram of the electronics within a preferred receiver which detects the complex coded message from the transmitter depicted in FIG. 4. The signal received is fed to a microcontroller 71 which decodes the signal. If the microprocessor finds that the decoded signal contains the module's address, the signal is passed on in electrical form to the microprocessor in the equipment console. The form of this output signal can be either a simple single electrical pulse for each received and correctly decoded message, or a message containing the current heart rate which has been calculated either in the transmitter or in the receiving module.

FIG. 8 is a block diagram of a preferred self-contained receiver/monitor which could be mounted, for example, within a wristwatch. Alternatively, other housings can be provided, such as a waist-worn module, a module mounted on the handlebars of a bicycle or the like in order to suit various exercise regimes. In the case of the preferred self-contained receiver/monitor, the 5 kHz radio signals detected in an aerial 81 and amplified by a receiver 82 are fed to a microcontroller 83 (exemplary of which is a Sanyo LC 5852N) which performs the functions of message decoding, optional heart rate calculation, heart rate display on a display unit 84, responding to user commands entered via keys 85, and like functions.

The dual frequency transmitters described in the embodiments above represent a simple low-cost upgrade to a conventional 5 kHz monitoring system which allows monitoring on conventional 5 kHz monitor/receivers as well as on HF or UHF monitoring systems used in gymnasia and in the region of electrically noisy pieces of exercise equipment. Users can therefore monitor heart rate while wearing their own transmitter both in a gymnasium and at home.

Various modifications may be made to the embodiments hereinbefore described without departing from the scope of the invention. For example, the transmitter may include a user actuatable switch to allow the user to disable the first signal (5 kHz) when on a treadmill, or the second signal (122 kHz) when outdoors, if required. This saves power and prolongs transmitter battery life. The receiver may include both 5 kHz and 122 kHz receiving circuits and the receiver may be set to select the 5 kHz or the high frequency signal 122 kHz depending on which signal shows the least interference.

We claim:

1. A transmitter for heart rate monitoring, said transmitter having detection means for detecting a heart rate signal, and heart rate signal processing means for processing the detected heart rate signal into a processed signal, and transmitting means for transmitting a first radio signal having a frequency about 5 kHz and for transmitting a second radio signal at a frequency substantially greater than 5 kHz, said first and said second radio signals corresponding to said processed signal.

2. A transmitter as claimed in claim 1 wherein the frequency of the second radio signal is at least 100 kHz.

3. A transmitter as claimed in claim 1 wherein the first and second radio signals are transmitted from respective first and second transmitter output stages simultaneously.

4. A transmitter as claimed in claim 1 wherein the first and second radio signals are successively transmitted.

5. A transmitter as claimed in claim 1 wherein the heart rate signal processing means includes a threshold detection circuit coupled to a pulse generator circuit for generating an output pulse when the processed signal exceeds a preset amplitude, said output pulse fed to said 5 kHz transmitter output stage and to a high frequency transmitter output stage for emitting said first and said second radio signals simultaneously.

6. A transmitter as claimed in claim 1 wherein the heart rate signal processing means includes a threshold detection circuit coupled to a microcontroller for generating an output pulse when the processed signal exceeds a preset amplitude, said output pulse being passed to said 5 kHz transmitter output stage, and to a high frequency output stage for emitting said first and said second radio signals simultaneously.

7. A transmitter as claimed in claim 1 wherein said transmitter is mounted on a chestbelt.

8. A transmitter as claimed in claim 1 wherein the transmitter includes switch means for allowing a user to deselect transmission of the first or the second radio signal.

9. A receiver for receiving first and second radio signals from a transmitter for heart rate monitoring, said first radio signal being about 5 kHz and said second radio signal being substantially greater than 5 kHz, said receiver having radio receiver means for receiving said first and second radio signals, and decoding means coupled to said receiving means for providing at least an output pulse for each correctly received signal.

10. A receiver as claimed in claim 9 wherein the decoding means is a microcontroller for decoding a coded message and for providing a decoded output signal, said decoded output signal being a single electrical pulse or a message containing the current heart rate.

11. A receiver as claimed in claim 9 wherein the decoding means is a decoding circuit for generating a single output pulse of a fixed duration for every correctly received signal.

12. A receiver as claimed in claim 9 wherein the receiver output is coupled to a microprocessor disposed in a console.

13. A receiver as claimed in claim 12 wherein the receiver is combined with the console.
